(19) United States
(12) Patent Application Publication
(10) Pub. No.: US 2016/0083374 A1
(43) Pub. Date: Mar. 24, 2016

(54) AMORPHOUS FORM OF CANAGLIFLOZIN AND PROCESS FOR PREPARING THEREOF

(71) Applicant: CADILA HEALTHCARE LIMITED, Gujarat (IN)

(72) Inventors: Shriprakash Dhar DWIVEDI, Gujarat (IN); Brij KHERA, Gujarat (IN); Jayprakash Ajitsingh PARIHAR, Gujarat (IN); Jagdish Maganlal PATEL, Gujarat (IN)

(21) Appl. No.: 14/891,654

(22) PCT Filed: May 30, 2014
(86) PCT No.: PCT/IN2014/000366

§ 371 Date: Nov. 16, 2015

(30) Foreign Application Priority Data
May 30, 2013 (IN) 1909/MUM/2013

(51) Int. Cl. C07D 409/10

(52) U.S. Cl. CPC C07D 409/10

(57) ABSTRACT

The present invention provides invention relates to stable amorphous form of Canagliflozin. The invention also provides the processes for the preparation of an amorphous form of Canagliflozin; and pharmaceutical compositions comprising therapeutically effective amount of an amorphous form of Canagliflozin, use of said composition for treatment of diabetes, obesity and diabetic complications, especially in type-2 diabetes.

### FIELD OF THE INVENTION

The invention relates to an amorphous form of canagliflozin. More particularly, it relates to processes for the preparation of an amorphous form of canagliflozin. The invention also relates to pharmaceutical compositions comprising therapeutically effective amount of an amorphous form of canagliflozin and use of said composition for treatment of diabetes, obesity and diabetic complications, especially in type-2 diabetes.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Canagliflozin is inhibitor of sodium dependent glucose transporter inhibitor (SGLT) which is chemically represented as (2S,3R,4R,5S,6R)-2-{3-[5-[4-Fluoro-phenyl]-thiophen-2-ylmethyl]-4-methyl-phenyl}-6-hydroxymethyl-tetrahydro-pyran-3,4,5-triol having structural Formula as represented by Formula (I).

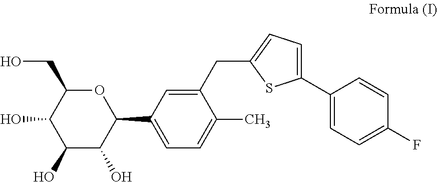 [SMILES: Cc1ccc([C@@H]2O[C@H](CO)[C@@H](O)[C@H](O)[C@H]2O)cc1Cc1ccc(-c2ccc(F)cc2)s1]

U.S. Pat. No. 7,943,788 B2 (the '788 patent) discloses canagliflozin or salts thereof and the process for its preparation.

U.S. Pat. Nos. 7,943,582 B2 and 8,513,202 B2 discloses crystalline form of 1-(β-D-glucopyranosyl)-4-methyl-3-[5-(4-fluorophenyl)-2-thienylmethyl]benzene hemihydrate and process for preparation thereof. The US 3 582 B2 and US '202 B2 further discloses that preparation of the crystalline form of hemi-hydrate canagliflozin typically involves dissolving in a good solvent (e.g. ketones or esters) crude or amorphous compound prepared in accordance with the procedures described in WO 2005/012326 pamphlet, and adding water and a poor solvent (e.g. alkanes or ethers) to the resulting solution, followed by filtration.

U.S. PG-Pub. No. 2013/0237487 A1 (the US '487 A1) discloses amorphous dapagliflozin and amorphous canagliflozin. The US '487 A1 also discloses 1:1 crystalline complex of canagliflozin with L-proline (Form CS1), ethanol solvate of a 1:1 crystalline complex of canagliflozin with D-proline (Form CS2), 1:1 crystalline complex of canagliflozin with L-phenylalanine (Form CS3), 1:1 crystalline complex of canagliflozin with D-proline (Form CS4).

The US '487 A1 discloses preparation of amorphous canagliflozin by adding its heated toluene solution into n-heptane. After drying in vacuo the product was obtained as a white solid of with melting point of 54.7° C. to 72.0° C. However, upon repetition of the said experiment, the obtained amorphous canagliflozin was having higher amount of residual solvents. Therefore, the amorphous canagliflozin obtained by process as disclosed in US '487 A1 is not suitable for pharmaceutical preparations.

The US '487 A1 further discloses that amorphous canagliflozin obtained by the above process is hygroscopic in nature which was confirmed by Dynamic vapor sorption (DVS) analysis. Further, it was observed that the amorphous form underwent a physical change between the sorption/desorption cycle, making the sorption/desorption behavior different between the two cycles. The physical change that occurred was determined to be a conversion or partial conversion from the amorphous state to a crystalline state. This change was supported by a change in the overall appearance of the sample as the humidity increased from 70% to 90% RH.

The canagliflozin assessment report EMA/718531/2013 published by EMEA discloses that Canagliflozin hemihydrate is a white to off-white powder, practically insoluble in water and freely soluble in ethanol and non-hygroscopic. Polymorphism has been observed for canagliflozin and the manufactured Form I is a hemihydrate, and an unstable amorphous Form II. Form I is consistently produced by the proposed commercial synthesis process.

Therefore, it is evident from the prior art that the reported amorphous form of canagliflozin is unstable and hygroscopic as well as not suitable for pharmaceutical preparations due to higher amount of residual solvents above the ICH acceptable limits.

Hence, there is a need to provide a stable amorphous form of canagliflozin which is suitable for pharmaceutical preparations.

Crystalline solids normally require a significant amount of energy for dissolution due to their highly organized, lattice like structures. For example, the energy required for a drug molecule to escape from a crystal is more than from an amorphous or a non-crystalline form. It is known that the amorphous forms in a number of drugs exhibit different dissolution characteristics and in some cases different bioavailability patterns compared to the crystalline form (Econno T., Chem. Pharm. Bull., 1990; 38: 2003-2007). For some therapeutic indications, one bioavailability pattern may be favoured over another.

An amorphous form of some of the drugs exhibit much higher bioavailability than the crystalline forms, which leads to the selection of the amorphous form as the final drug substance for pharmaceutical dosage from development. Additionally, the aqueous solubility of crystalline form is lower than its amorphous form in some of the drugs, which may resulted in the difference in their in vivo bioavailability. Therefore, it is desirable to have amorphous forms of drugs with high purity to meet the needs of regulatory agencies and also highly reproducible processes for their preparation.

In view of the above, it is therefore, desirable to provide canagliflozin amorphous form as well as an efficient, economical and eco-friendly process for the preparation of highly pure canagliflozin amorphous form.

### SUMMARY OF THE INVENTION

In one general aspect, there is provided a stable amorphous form of canagliflozin of Formula (I).

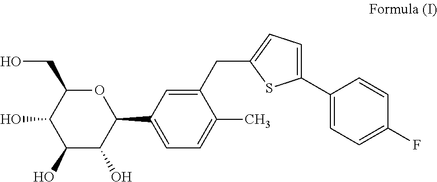 [SMILES: Cc1ccc([C@@H]2O[C@H](CO)[C@@H](O)[C@H](O)[C@H]2O)cc1Cc1ccc(-c2ccc(F)cc2)s1]

In another general aspect, there is provided a process for the preparation of stable amorphous form of canagliflozin.

In another general aspect, there is provided a process for preparation of stable amorphous form of canagliflozin comprising:
a) providing a solution of canagliflozin in one or more organic solvent; and
b) obtaining a stable amorphous form of canagliflozin by removal of the solvent.

In another general aspect, there is provided a process for preparing the amorphous form of canagliflozin, which comprises:
a) providing a solution of canagliflozin in one or more organic solvent;
b) adding anti-solvent to the solution; and
c) obtaining amorphous form of canagliflozin.

In another general aspect, there is provided a process for the preparation of stable amorphous form of canagliflozin comprising:
a) obtaining canagliflozin;
b) preparing a multicomponent composition of canagliflozin;
c) converting the multicomponent composition of canagliflozin of step b) into the canagliflozin;
d) dissolving the canagliflozin of step c) in one or more organic solvent; and
e) obtaining the stable amorphous form of canagliflozin by removal of the organic solvent.

In general aspect, there is provide an amorphous solid dispersion of canagliflozin and a polymer.

In another general aspect, there is provided a pharmaceutical composition comprising an amorphous form of canagliflozin. In particular, the pharmaceutical composition is an amorphous solid dispersion of canagliflozin and a polymer.

In another general aspect, there is provided a pharmaceutical composition comprising a stable amorphous canagliflozin and one or more pharmaceutically acceptable carriers, excipients or diluents.

### BRIEF DESCRIPTION OF THE ACCOMPANYING DRAWINGS

FIG. 1. Shows x-ray diffractogram (XRD) of amorphous form of canagliflozin.

FIG. 2. Shows x-ray diffractogram (XRD) of amorphous form of canagliflozin.

### DETAILED DESCRIPTION OF THE INVENTION

The above and other objects of the present invention are achieved by the process of the present invention, which leads to a stable amorphous form of canagliflozin suitable for pharmaceutical preparations and having greater stability. The invention also provides a process for preparing a stable amorphous form of canagliflozin using an organic solvent.

As used herein, the term “a multicomponent composition” means a solid composition containing canagliflozin and another component which forms a co-crystal or co-precipitate with canagliflozin, or hydrates or solvates thereof.

As used herein, the term “stable amorphous canagliflozin” includes amorphous canagliflozin that does not convert to any other solid form when stored at a temperature of up to. about 40° C. and at, a relative humidity of about 25% to about 75% for about three months or more.

As used herein, the term “solid dispersion” means any solid composition having at least two components. In certain embodiments, a solid dispersion as disclosed herein includes an active ingredient canagliflozin dispersed among at least one other component, for example a polymer.

The term “immobilize” as used herein with reference to the immobilization of the active compound i.e. canagliflozin in the polymer matrix, means that molecules of the active compound interact with molecules of the polymer in such a way that the molecules of the canagliflozin are held in the aforementioned matrix and prevented from crystal nucleation due to lack of mobility.

As used herein, the terms “obtaining” means isolating the amorphous form of canagliflozin by way of filtration, filtration under vacuum, centrifugation, decantation. The product obtained may be further or additionally dried to achieve the desired moisture values. For example, the product may be dried in a tray drier, dried under vacuum and/or in a Fluid Bed Drier.

In one general aspect, there is provided a stable amorphous form of canagliflozin of Formula (I).

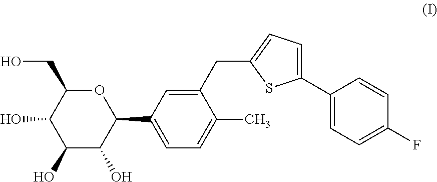 [SMILES: Cc1ccc([C@@H]2O[C@H](CO)[C@@H](O)[C@H](O)[C@H]2O)cc1Cc1ccc(-c2ccc(F)cc2)s1]

In another general aspect, there is provided an amorphous form of canagliflozin having purity by HPLC greater than 99% and residual solvents less than 0.5%.

In general, the amorphous form of canagliflozin is substantially free from residual solvents. The term “substantially free” means residual solvents within the permissible ICH limits suitable for pharmaceutical preparations. For example but not limited to less than 0.5%, particularly less than 0.3% or more particularly less than 0.2%.

In another general aspect, there is provided a process for the preparation of a stable amorphous form of canagliflozin comprising:
a) providing a solution of canagliflozin in one or more organic solvent; and
b) obtaining the stable amorphous form of canagliflozin by removal of the organic solvent.

In general, the organic solvent of step a) comprises one or more of alcohol, ketone, ester, hydrocarbon, acetonitrile, or mixtures thereof. In particular, methanol, ethanol, isopropanol, 2-propanol, 1-butanol, t-butyl alcohol, 1-pentanol, 2-pentanol, amyl alcohol, ethylene glycol, glycerol, acetone, butanone, 2-pentanone, 3-pentanone, methylbutyl ketone, methyl isobutyl ketone, ethyl acetate, propyl acetate, isopropyl acetate, t-butyl acetate, isobutyl acetate, toluene, xylene, methylene dichloride, ethylene dichloride, chlorobenzene, acetonitrile. More particularly, acetone, toluene, ethyl acetate or methylene dichloride may be used.

Step b) involves removal of the solvent to obtain an amorphous form of canagliflozin. The isolation may be affected by removing solvent. Techniques which may be used for the removal of solvent include distillation, distillation under vacuum, spray drying, agitated thin film dyring (“ATFD”), and freeze drying (lyophilization).

The obtained amorphous canagliflozin is stable under normal stability conditions and substantially free from residual solvent. Therefore, there is no physical change observed from amorphous form to crystalline form during the stability.

In one preferred embodiment of the invention, the solution of canagliflozin in one or more of organic solvents may be spray dried, which involves the spray drying of feed stock, which may be prepared as discussed below. In general, the feedstock is dozed into the spray-dryer under the following parameters.

| Sr. No. | Parameters | Conditions | |
|---|---|---|---|
| a) | Feed pump | 10-50 | rpm |
| b) | Inlet temperature | 35°-80° | C. |
| c) | Outlet temperature | 30°-60° | C. |
| d) | Aspirator rate | 1000-1500 | rpm |
| e) | Vacuum for conveying the | 30-120 | mm of Hg |
| | dry product | | |
| f) | Hot air supply | 2-4 | Kg/cm² |
| g) | Atomizer Speed: | 40,000-100,000 | rpm |

In a preferred feature, the feedstock for spray drying is either a clear solution or in dispersion form.

According to further general aspect, canagliflozin may be spray dried by dissolving or suspending or slurring in one or more organic solvents to obtain amorphous form. In the present invention, the feed stock of canagliflozin in organic solvent is spray-dried. Thus obtain spry-dried compound was in amorphous form, this fact was again confirmed by the X-ray powder diffractogram of spray-dried canagliflozin.

In a specific preferred embodiment of the invention, weighed quantity of canagliflozin was dissolved in 2-10 volumes of chosen solvent, preferably 4-5 volumes solvent at 25° C. to 30° C. The content was stirred for 30 minutes at 25° C. to 30° C. The content was filtered through Hyflosupercell, and filtrate was spray dried under above conditions. The obtained powder was further dried at 40° C. for 12-16 hours under vacuum to afford the stable amorphous form of canagliflozin.

In the present invention, feed stock of canagliflozin was conveniently prepared by dissolving canagliflozin in one or more of organic solvent selected from acetone, C₁₋₄alcohol, C₂₋₆acetate, acetonitrile, methylene dichloride or mixture thereof. In particular, methanol, ethanol, acetone, ethyl acetate, methylene dichloride are solvents used or such solvents that evaporate easily to afford dry product. More particularly, methanol, acetone, ethanol, ethyl acetate or mixtures of the above.

In another general aspect, there is provided a process for preparing the amorphous form of canagliflozin, which comprises:
a) providing a solution of canagliflozin in one or more of organic solvent;
b) adding anti-solvent to the solution; and
c) obtaining amorphous form of canagliflozin.
   Step a) involves providing a solution of canagliflozin in a solvent or mixture of solvent. The solution for step a) can be obtained by the known methods that include:
(i) direct use of a reaction mixture containing canagliflozin that is obtained in the course of its synthesis; or
(ii) dissolving canagliflozin in one or more of organic solvent.
   The organic solvent comprises one or more of solvents as defined herein above. In particular toluene or ethyl acetate may be used.

The anti-solvent for step b) comprises one or more of hexanes, n-heptane, n-pentane, cyclohexane, methylcyclohexane; diethyl ether, diisopropyl ether, t-butyl methyl ether, dibutyl ether, tetrahydrofuran, 1,4-dioxane, 2-methoxy ethanol. In particular, cyclohexane, n-hexane or n-heptane may be used.

Step c) involves obtaining of the amorphous form of canagliflozin, which may be affected by removing solvent using techniques which may be used for the removal of solvent include as defined herein above.

In another general aspect, there is provided a process for the preparation of stable amorphous form of canagliflozin, comprising:
a) obtaining canagliflozin;
b) preparing a multicomponent composition of canagliflozin;
c) converting the multicomponent composition of canagliflozin of step b) into the canagliflozin;
d) dissolving the canagliflozin of step c) in one or more organic solvent; and e) obtaining stable amorphous form of canagliflozin by removal of organic solvent.

The multicomponent composition of the present invention comprises one or more of hydrates, solvates, co-crystals or co-precipitate or crystalline complex of canagliflozin or hydrate or solvates thereof.

In general, the multicomponent composition may be selected from canagliflozin-L-proline crystalline complex, canagliflozin-D-proline crystalline complex, ethanol solvate of canagliflozin-D-proline crystalline complex, canagliflozin-citric acid co-crystal, canagliflozin-L-phenylalanine crystalline complex or hydrates or solvate forms thereof. In particular, canagliflozin-L-proline crystalline complex may be prepared.

In general, the process further comprises converting canagliflozin-L-proline crystalline complex to canagliflozin and thereby to its amorphous form.

In general, the organic solvent for preparing a multicomponent composition of canagliflozin comprises one or more of methanol, ethanol, isopropanol, acetone, methyl ethyl ketone, methylisobutyl ketone, ethyl acetate, isopropyl acetate, methylenedichloride, ethylene dichloride, acetonitrile or mixtures thereof.

The obtained multicomponent composition may be converted to amorphous form of canagliflozin by the treating the crystalline complex with base to obtain canagliflozin. The obtained canagliflozin may be converted to amorphous form by any of the process disclosed herein with or without isolation of canagliflozin.

In general, the base comprises one or more of sodium hydroxide, potassium hydroxide, lithium hydroxide, calcium hydroxide, barium hydroxide, sodium carbonate, potassium carbonate, sodium bicarbonate, potassium bicarbonate. In particular sodium hydroxide or potassium hydroxide may be used.

In general, the stable amorphous form of canagliflozin is obtained by removal of solvent. The removal of solvent comprises one or more of distillation, distillation under vacuum, spray drying, agitated thin film dyring (“ATFD”), and freeze drying (lyophilization).

In another general aspect, there is provided amorphous solid dispersion of canagliflozin and a polymer.

In another general aspect, there is provided a composition comprising an amorphous form of canagliflozin. In particular, the composition is an amorphous solid dispersion of canagliflozin and a polymer.

In general, the polymer may be a non-ionic polymer or an ionic polymer. The polymer comprises one or more of hydroxypropylmethyl cellulose acetate succinate (HPMC-AS), hydroxypropylmethyl cellulose (HPMC), methacrylic acid copolymers, polyvinyl pyrrolidone (PVP). In particular, PVP of different grades like K-15, K-30, K-60, K-90 and K-120 may be used for the preparation of amorphous composition. In particular, HPMC-AS and PVP K-30 may be used.

In some embodiments, the canagliflozin of Formula (I) may be dispersed within a matrix formed by a polymer in its solid state such that it is immobilized in its amorphous form. The polymer may prevent intramolecular hydrogen bonding or weak dispersion forces between two or more drug molecules of canagliflozin. The solid dispersion provides for a large surface area, thus further allowing for improved dissolution and bioavailability of canagliflozin.

In general, the ratio of the amount of weight of canagliflozin within the solid dispersion to the amount by weight of the polymer therein is from about 1:1 to about 1:10. The composition of canagliflozin with polymer, preferably PVP K-30 or HPMC-AC may be prepared by using about 1:1 to about 1:10 polymers with respect to canagliflozin. The usage of higher molar amount of polymer increases the amorphous character of the drug substance.

In another general aspect, there is provided a process for the preparation of composition of an amorphous solid dispersion of canagliflozin, the process comprising mixing canagliflozin with a polymer in one or more organic solvent and obtaining the amorphous solid dispersion of canagliflozin by the removal of the solvent.

The canagliflozin and a polymer (for example HPMC-AS or PVP K-30) may be dissolved in one or more of organic solvent having a low boiling point, e.g. methanol, ethanol, isopropanol, acetone, ethyl acetate, water or mixtures thereof. The amorphous solid dispersion may be obtained by removal of solvent (for example by evaporation, evaporation under reduced pressure, spray drying, lyophilization, flash evaporation, vacuum distillation) thereby leaving the amorphous solid dispersion precipitated in a matrix formed by the polymer.

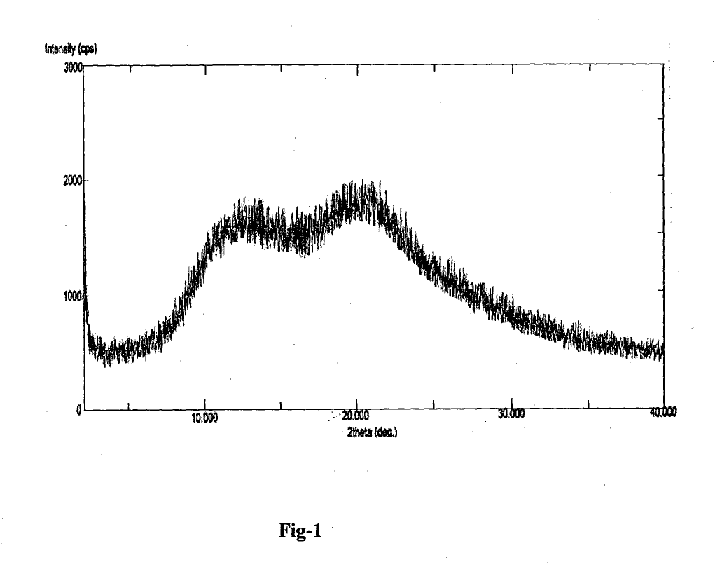

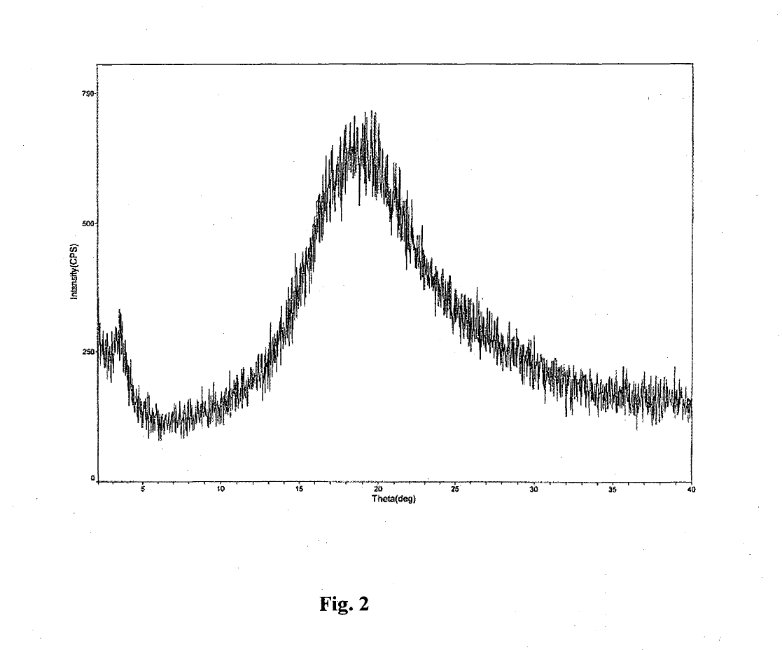

In another general aspect, the amorphous form of canagliflozin is characterized by X-ray powder diffraction as depicted in FIG. 1 and FIG. 2.

In another general aspect, there is provided a stable amorphous form of canagliflozin which is stable during storage and drying.

In another general aspect, the stable amorphous form of canagliflozin, is stored under nitrogen atmosphere and packed in a double polythene bag tied with a thread, keeping primary packing containing amorphous canagliflozin or salts thereof inside a black color polyethylene bag containing oxygen busters and sealing it, placing above the double polyethylene bag inside a triple laminated bag optionally containing oxygen busters and sealing it, and placing the sealed triple laminated bag inside a closed high density polyethylene (HDPE) container and storing in controlled environment chamber at about 25° C. and/or 40° C.

In another general aspect, the present invention provides an amorphous form of canagliflozin having purity by HPLC of >98%. In particular, the purity by HPLC of >99%, more particularly, the purity by HPLC of >99.5%, further more particularly, the purity by HPLC of >99.8%, most particularly, the purity by HPLC >99.9%.

Powder X-ray Diffraction of amorphous canagliflozin ⁻can be obtained under following conditions.

Powder X-ray Diffraction: X-ray powder diffraction spectrum was observed on a MF 2100 2 KW X-ray Powder diffractometer of make Rigaku or equivalent having a Copper Kα-radiation at a voltage of 40 kV and 30 mA. Approximately 150 mg sample was gently flattened on a quartz plate without further processing (e.g. Grinding and sieving) and scanned from 4° to 40° at 0.010° sampling width and 4.000° per minute.

In another general aspect, canagliflozin to be used as the starting material can be prepared by the known methods reported in the prior i.e. by using the process as per U.S. Pat. No. 7,943,788 B2, which is incorporated herein as reference in its entirety.

In another general aspect, there is provided a pharmaceutical composition comprising a stable amorphous canagliflozin and one or more pharmaceutically acceptable carriers, excipients or diluents.

In another general aspect, there is provided a pharmaceutical composition comprising an amorphous solid dispersion of canagliflozin and a polymer together with one or more of pharmaceutically acceptable carriers, excipients or diluents.

The invention also encompasses pharmaceutical compositions comprising canagliflozin or salts thereof of the invention. As used herein, the term “pharmaceutical compositions” or “pharmaceutical Formulations” includes tablets, pills, powders, liquids, suspensions, emulsions, granules, capsules, suppositories, or =injection preparations.

Pharmaceutical compositions containing the canagliflozin of the invention may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, liquids, suspensions, emulsions, granules, capsules, suppositories, or injection preparations.

Having described the invention with reference to certain preferred embodiments, other embodiments will become apparent to one skilled in the art from consideration of the specification.

The process for preparation of the amorphous form of canagliflozin is demonstrated in examples illustrated below. These examples are provided as illustration only and therefore should not be construed as limitation of the scope of invention.

### EXAMPLE—1

Preparation of amorphous form of Canagliflozin

In 100 ml three necked round bottomed flask equipped with mechanical stirrer, thermometer and addition funnel, 25.0 g of canagliflozin was dissolved in 250.0 mL of methanol mixture at 25° C. to 30° C. The content was stirred for 30 minutes at 25° C. to 30° C. To this, 1.0 g charcoal was added and stirred for 30 minutes at 25° C. to 30° C. The content was filtered through Hyflo-supercel, and the Hyflo-supercel pad was washed with 50.0 mL methanol. The filtrate was concentrated under vacuum below 45° C. followed by spray drying in JISL Mini spray drier LSD-48 under the below conditions. The product was collected from cyclone and is further dried at 55° C±5° C. under vacuum for 16 hours to get 19.0 g of amorphous canagliflozin.

| Sr. No. | Parameters | Conditions | |
|---|---|---|---|
| a) | Feed pump | 30 | rpm |
| b) | Inlet temperature | 60° | C. |
| c) | Outlet temperature | 40° | C. |
| d) | Aspirator rate | 1300 | rpm |
| e) | Vacuum for conveying the dry product | 80 | mm of Hg |
| h) | Hot air supply | 2 | Kg/cm² |

The spray-dried canagliflozin is amorphous in nature. The obtained product contains residual solvent well within ICH limit.

The obtained solid was amorphous canagliflozin as is shown by the X-ray diffraction pattern shown in FIG. 1.

### EXAMPLE—2

Preparation of Amorphous form of Canagliflozin

In 100 ml three necked round bottomed flask equipped with mechanical stirrer, thermometer and addition funnel, 25.0 g of canagliflozin was dissolved in 250.0 mL of acetone mixture at 25° C. to 30° C. The content was stirred for 30 minutes at 25° C. to 30° C. To this, 1.0 g charcoal was added and stirred for 30 minutes at 25° C. to 30° C. The content was filtered through Hyflo-supercel, and the Hyflo-supercel pad was washed with 50.0 mL acetone. The filtrate was concentrated under vacuum below 45° C. followed by spray drying in JISL Mini spray drier LSD-48 under the below conditions. The product was collected from cyclone and is further dried at 55° C.±5° C. under vacuum for 16 hours to get 20.0 g of amorphous canagliflozin.

| Sr. No. | Parameters | Conditions | |
|---|---|---|---|
| a) | Feed pump | 30 | rpm |
| b) | Inlet temperature | 60° | C. |
| c) | Outlet temperature | 40° | C. |
| d) | Aspirator rate | 1300 | rpm |
| e) | Vacuum for conveying the dry product | 80 | mm of Hg |
| h) | Hot air supply | 2 | Kg/cm² |

The spray-dried canagliflozin is amorphous in nature. The compound is having residual acetone less than 0.5% by GC.

The obtained solid was amorphous canagliflozin as is shown by the X-ray diffraction pattern shown in FIG. 2.

### EXAMPLE—3

Preparation of Amorphous form of Canagliflozin

In 100 ml three necked round bottomed flask equipped with mechanical stirrer, thermometer and addition funnel, 10 g of canagliflozin was dissolved in 125 mL methanol and heated to obtain clear solution at 65° C. The solution was distilled to remove methanol completely. The compound thus obtained was amorphous canagliflozin.

### EXAMPLE—4

Preparation of Amorphous form of Canagliflozin

In 100 ml three necked round bottomed flask equipped with mechanical stirrer, thermometer and addition funnel, 10 g of canagliflozin was dissolved in 125 mL acetone and heated to obtain clear solution at 65° C. The solution was distilled to remove acetone completely. The compound thus obtained was amorphous canagliflozin. The compound is having residual acetone less than 0.5% by GC.

### EXAMPLE 5

Preparation of Amorphous form of Canagliflozin

In 100 ml three necked round bottom flask equipped with mechanical stirrer, thermometer and an addition funnel, canagliflozin (0.5 gm, 1.02 mmol), PVP K-30 (4 gm, 8 times) and 88% methanol in water (12.5 ml, 25V) were heated to 65-70° C. to get clear solution. The reaction mixture was stirred for 1 hour, concentrated under vacuum (1.5 mbar) at 65-70° C. and degassed under vacuum (1.5 mbar) for 1 hour at 70° C. to obtain the title compound in amorphous form.

### EXAMPLE 6

Preparation of Amorphous form of Canagliflozin

In 100 ml three necked round bottom flask equipped with mechanical stirrer, thermometer and an addition funnel, canagliflozin (0.5 gm, 1.02 mmol), I-IPMC-AS (1 gm, 2 times) in 88% methanol in water (12.5 ml, 25V) were heated at 65 to 70° C. to get clear solution. The reaction mixture was stirred for 2 hours, concentrated under vacuum (1.5 mbar) at 70° C. and degassed under vacuum (1.5 mbar) for 1 hr at 70° C. to obtain the title compound in amorphous form.

### EXAMPLE—7

### Preparation of Canagliflozin-L-Proline Crystalline Complex

In 100 ml three necked round bottomed flask equipped with mechanical stirrer, thermometer and addition funnel; 25.0 g of canagliflozin, 6.06 g L-proline and 250 mL ethanol were heated to 75-80° C., stirred for 15 min and then cooled down to 25-30° C. The mass was filtered and washed with ethanol to obtain canagliflozin-L-proline crystalline complex.

### EXAMPLE—8

Preparation of Amorphous Canagliflozin from Canagliflozin-L-Proline Crystalline Complex

In 100 ml three necked round bottomed flask equipped with mechanical stirrer, thermometer and addition funnel 25.0 g of Canagliflozin-L-Proline Crystalline Complex and 250 mL of ethyl acetate were stirred to get a clear solution, washed with 2×150 mL of water and the organic layer was distilled. To the residue 100 mL of isopropyl acetate and 2.5 mL of water was added and heated to 75-80° C., stirred for 15 min and cooled down to 25-30° C. The mass filtered and washed with isopropyl acetate to obtain canagliflozin. The obtained canagliflozin was subjected to spray dyring under conditions of example-2 using acetone solvent to obtain amorphous canagliflozin. Purity >99.5% by HPLC. The compound is having residual acetone less than 0.5% by GC.

The obtained solid was amorphous canagliflozin as shown by the X-ray diffraction pattern shown in FIG. 2.

HPLC Purity of Amorphous Canagliflozin was Measured by using Following Chromatographic Conditions:
Equipment: Shimadzu LC2010C HPLC system equipped with a dual wavelength UV-VIS detector or equivalent
Column: Kromasil C-8 (250 mm×4.6 mm, 5 gm) or equivalent
Flow rate: 1.5 mL/minute
Column oven temp.: 30° C.
Wavelength: 210 nm
Injection Volume: 10 μL
Diluent: Mobile Phase A: Mobile Phase B (30:70)
Mobile Phase A: Buffer:Acetonitrile:Methanol (60:30:10)
Mobile Phase B: Acetonitrile: Methanol (80:20)

### EXAMPLE—9

Preparation of Amorphous form of Canagliflozin as per Example—2 of US '487 A1

In 100 ml three necked round bottomed flask equipped with mechanical stirrer, thermometer and addition funnel 25.0 g of canagliflozin and 150 mL of ethyl acetate were stirred to get clear solution. 100 mL of n-heptane was added to the solution and the reaction mixture was filtered and dried to obtain amorphous canagliflozin. The obtained amorphous canagliflozin were dried at 65° C. under vacuum for 72 hours. The residual n-heptane was 44000 ppm by GC after 72 hours drying.

### EXAMPLE—10

Replacing Toluene with Ethyl Acetate in above Example—9

In 100 ml three necked round bottomed flask equipped with mechanical stirrer, thermometer and addition funnel 25.0 g of canagliflozin and 150 mL of ethyl acetate were stirred to obtain clear solution. 100 mL of n-heptane was added to the solution and the reaction mixture was filtered and dried to obtain amorphous canagliflozin. The obtained amorphous canagliflozin were dried at 65° C. under vacuum for 72 hours. The residual n-heptane was 44000 ppm by, GC after 72 hours drying.

### EXAMPLE—11

Replacing n-Heptane with Cyclohexane in above Example—9

In 100 ml three necked round bottomed flask equipped with mechanical stirrer, thermometer and addition funnel 25.0 g of canagliflozin and 150 mL of ethyl acetate were stirred to obtain clear solution. 100 mL of cyclohexane was added to the solution and the reaction mixture was filtered and dried to obtain amorphous canagliflozin. The obtained amorphous canagliflozin were dried at 55° C. under vacuum for 72 hours. The residual cyclohexane was >5000 ppm by GC after 72 hours drying.

### EXAMPLE—12

Preparation of Amorphous form of Canagliflozin

In 100 ml three necked round bottomed flask equipped with mechanical stirrer, thermometer and addition funnel; 25.0 g of canagliflozin and 250 mL of ethyl acetate were stirred to get clear solution and then ethyl acetate was removed under reduced pressure to obtain 20.0 g of amorphous canagliflozin. The obtained amorphous canagliflozin were dried at 55° C. under vacuum for 72 hours. The residual ethyl acetate was ˜8450 ppm by GC after 72 hours drying.

While the present invention has been described in terms of its specific embodiments, certain modification and equivalents will be apparent to those skilled in art and the intended to be included within the scope of the invention.

We claim:

1. A stable amorphous form of Canagliflozin of Formula (I) 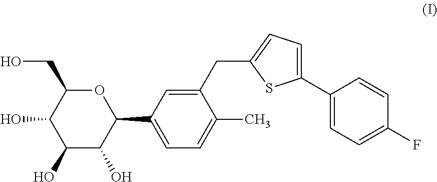 [SMILES: Cc1ccc([C@@H]2O[C@H](CO)[C@@H](O)[C@H](O)[C@H]2O)cc1Cc1ccc(-c2ccc(F)cc2)s1]
wherein the amorphous Canagliflozin does not convert to any other solid form when stored at a temperature of up to about 40° C. and at a relative humidity of about 25% to about 75% for about three months or more.

2. An amorphous form of Canagliflozin having purity by HPLC greater than 99% and residual solvent less than 0.5%.

3. The process for the preparation of stable amorphous form of Canagliflozin as claimed in claim 1, the process comprising:
a) providing a solution of Canagliflozin in one or more organic solvent; and
b) obtaining the stable amorphous form of Canagliflozin by the removal of the solvent.

4. The process as claimed in claim 3, wherein the organic solvent comprises one or more of alcohol, ketone, ester, hydrocarbon, acetonitrile, or mixtures thereof.

5. The process as claimed in claim 4, wherein the alcohol comprises one or more of methanol, ethanol, isopropanol, 2-propanol, 1-butanol, t-butyl alcohol, 1-pentanol, 2-pentanol, amyl alcohol, ethylene glycol, and glycerol.

6. The process as claimed in claim 4, wherein the ketone comprises one or more of acetone, butanone, 2-pentanone, 3-pentanone, methylbutyl ketone, and methyl isobutyl ketone.

7. The process as claimed in claim 4, wherein the ester comprises ethyl acetate, propyl acetate, isopropyl acetate, t-butyl acetate, and isobutyl acetate.

8. The process as claimed in claim 4, wherein the hydrocarbon comprises one or more of toluene, xylene, methylene dichloride, ethylene dichloride, and chlorobenzene.

9. The process as claimed in claim 3, wherein removing the solvent comprises one or more of distillation, distillation under vacuum, spray drying, agitated thin film drying (“ATFD”), and freeze drying (lyophilization).

10. The process for the preparation of stable amorphous form of canagliflozin as claimed in claim 1, the process comprising:
a) obtaining Canagliflozin;
b) preparing a multicomponent composition of Canagliflozin;
c) converting the multicomponent composition of Canagliflozin of step b) into Canagliflozin;
d) dissolving the Canagliflozin of step c) in one or more organic solvent; and
e) obtaining the stable amorphous form of Canagliflozin by the removal of the organic solvent.

11. The process as claimed in claim 10, wherein the multicomponent composition of Canagliflozin comprises one or more of Canagliflozin-L-proline crystalline complex, Canagliflozin-D-proline crystalline complex, ethanol solvate of Canagliflozin-D-proline crystalline complex, Canagliflozin-citric acid co-crystal, Canagliflozin-L-phenylalanine crystalline complex or hydrated or solvated forms thereof.

12. The process as claimed in claim 10, wherein removing the solvent comprises one or more of distillation, distillation under vacuum, spray drying, agitated thin film dyring (“ATFD”), and freeze drying (lyophilization).

13. An amorphous solid dispersion of Canagliflozin and a polymer.

14. The amorphous solid dispersion as claimed in claim 13, wherein the polymer is a non-ionic polymer or an ionic polymer.

15. The amorphous solid dispersion as claimed in claim 14, wherein the polymer comprises one or more of HPMC-AS, HPMC, methacrylic acid copolymers, and PVP.

16. The process for the preparation of an amorphous solid dispersion of Canagliflozin as claimed in claim 13, the process comprising mixing Canagliflozin with a polymer in one or more organic solvent and obtaining the amorphous solid dispersion of Canagliflozin by the removal of the solvent.

17. The process as claimed in claim 16, wherein the organic solvent comprises one or more of methanol, ethanol, isopropanol, acetone, ethyl acetate, water, or mixtures thereof.

18. The amorphous solid dispersion as claimed in claim 13, which is substantially free from crystalline forms and residual solvents.

19. An amorphous form of Canagliflozin having purity by HPLC of >98%.

20. A pharmaceutical composition comprising a stable amorphous Canagliflozin and one or more pharmaceutically acceptable carriers, excipients or diluents.

21. A pharmaceutical composition comprising an amorphous solid dispersion of Canagliflozin and a polymer together with one or more of pharmaceutically acceptable carriers, excipients or diluents.
